(19) United States
(12) Patent Application Publication
(10) Pub. No.: US 2009/0198058 A1
(43) Pub. Date: Aug. 6, 2009

(54) Process for Preparation of Dextrorotatory Isomer of 6-(5- chloro-pyrid-2-yl)-5-[(4-methyl -1-piperazinyl) carbonyloxy] -7-oxo-6,7-dihydro-5H-pyrrolo [3,4-b] pyrazine (Eszopiclone)

(76) Inventors: Dhananjay Govind SATHE, Thane (IN); Nandu Baban Bhise, Mumbai (IN); Harish Kashinath Mondkar, Mumbai (IN); Anand Vinod Shindikar, Aurangabad (IN); Manoj Madhukarrao Deshpande, Ahmedpur (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/186,567

(22) Filed: Aug. 6, 2008

(30) Foreign Application Priority Data
Aug. 6, 2007 (IN) 1511/MUM/2007
Aug. 6, 2008 (IN) PCT/IN2008/000487

(51) Int. Cl. C07D 487/04

(52) U.S. Cl. 544/350

(57) ABSTRACT

Disclosed herein is the process for preparation of 6-(5-chloro-pyrid-2-yl)-5-[(4-methyl-1-piperazinyl)carbonyloxy]-7-oxo-6,7-dihydro-5H-pyrrolo[3,4-b]pyrazine (Zopiclone), its resolution to get the dextrorotatory isomer of formula (I) substantially free of R(−) enantiomer and recovery of key raw material i.e. 6-(5-chloro pyrid-2-yl)-5-hydroxy-7-oxo-5,6-dihydropyrrolo[3,4-b]pyrazine from the R-isomer of Zopiclone followed by conversion of the recovered compound to get pure Eszopiclone (I) in high yield and high purity.

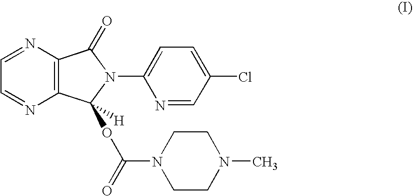

### RELATED APPLICATION

This application is claiming priority from Indian Patent Application number 1511/MUM/2007 filed 6 Aug. 2007, the contents of which are incorporated by reference here.

### TECHNICAL FIELD

The present invention relates to process for preparation of dextrorotatory isomer of 6-(5-chloro-pyrid-2-yl)-5-[(4-methyl-1-piperazinyl)carbonyloxy]-7-oxo-6,7-dihydro-5H-pyrrolo[3,4-b]pyrazine (Eszopiclone) of Formula (I), a hypnotic agent. The invention further relates to recovery of key starting material (IV) from unwanted R-isomer and converting into racemic Zopiclone and further converting into Eszopiclone (I).

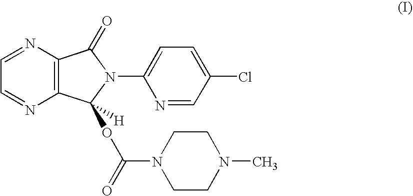

### BACKGROUND AND PRIOR ART

Zopiclone, chemically named as (±)-6-(5-chloro-pyrid-2-yl)-5-[(4-methyl-1-piperazinyl) carbonyloxy]-7-oxo-6,7-dihydro-5H-pyrrolo[3,4-b]pyrazine is a non-benzodiazepine with hypnotic activity. Although chemically unrelated to the benzodiazepines, Zopiclone possesses a spectrum of activity analogous to that of benzodiazepines (Goa, K. L. & Heel, R. C., Drugs, 32:48-65 (1986)). Zopiclone and its optically pure enantiomers are reportedly useful in the treatment of disease conditions including epilepsy, anxiety, aggressive behavior, muscle tension, behavioral disorders and depression. Recent studies (Chirality 1993, vol. 5, 419) have confirmed that the dextrorotatory enantiomer of Zopiclone is approximately twice as active as the racemate whereas the levorotatory isomer is almost inactive. Moreover, according to EP0609210 the levorotatory isomer is responsible for the majority of adverse effects which are associated with the administration of the medicine. Therefore, it is much more convenient to use the optically active pure S-enantiomer of Zopiclone than the racemate.

Eszopiclone, marketed by Sepracor under the brand Lunesta™ is a stereoselective isomer of Zopiclone. Sepracor (the originator of Eszopiclone) has stated that the drug acts rapidly with the duration of effect lasting upto 6 hours and used for the treatment of insomnia. The recommended dosing to improve sleep onset and maintenance is 2 mg for adult patients. Clinical studies demonstrate no evidence of tolerance in any patients and also following discontinuation, there was no rebound effect observed compared with baseline on sleep maintenance. Eszopiclone produced significant improvements in sleep, decreased nocturnal awakening due to hot flashes and positively affected mood in peri-menopausal and menopausal women.

U.S. Pat. No. 3,862,149 (referred hereinafter as US '149) discloses synthesis of Zopiclone wherein solution of 6-(5-chloropyrid-2-yl)-5-hydroxy-7-oxo-5,6-dihydropyrrolo[3,4-b]pyrazine (IV) in dimethylformamide (DMF) is added to suspension of sodium hydride (in molar ratio of 1:1) in DMF. To this a solution of N-methyl piperazine carbonyl chloride (III) in DMF is added and the obtained product is subjected to column chromatography using ethyl acetate:methanol and further recrystallized with acetonitrile:diisopropylether to get racemic Zopiclone (V) in low yield. This patent involves use of column chromatography for purifying the product which is not industrially feasible. Equivalent molar ratio of sodium hydride and compound (IV) used leads to incomplete reaction. Mode of addition of reagents such as addition of compound (IV) to sodium hydride suspension results in low yield of the product and increased formation of impurities.

The process described in US '149 uses excess of solvents, results in poor yield of the final compound and increases the time period for completing the reaction. Moreover, the final product in the process was purified using column chromatography and a mixture of solvents for recrystallization. These solvents are difficult to recover which makes the process unsuitable for use on a commercial scale. The reaction is represented by the scheme 1 as below.

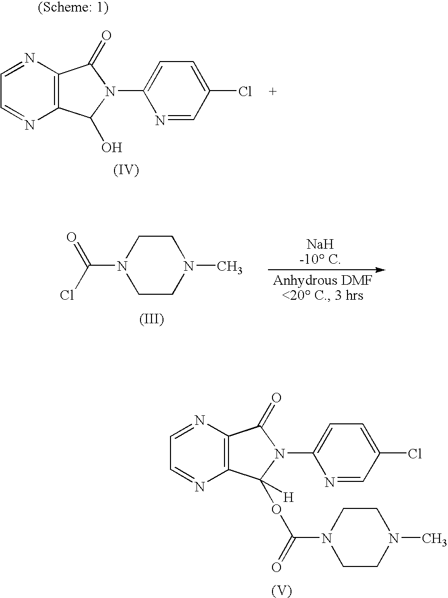

WO9212980 (equivalent to U.S. Pat. No. 6,444,673) discloses dextrorotatory isomer of Zopiclone (I) wherein racemic Zopiclone (V) is treated with D(+)-O,O-dibenzoyl tartaric acid monohydrate (VI) in dichloromethane (MDC) followed by evaporation of dichloromethane to get the compound (VII) which is further purified twice with dichloromethane:acetonitrile which gives purified D(+)-O,O-dibenzoyl tartarate salt of (S)-Zopiclone (VIII). Purified tartarate salt of Eszopiclone on basification using 2N aqueous sodium hydroxide gives Eszopiclone in crude state (IX) which on crystallization with acetonitrile gives Eszopiclone pure (I) (yield-23%). The patent discloses use of large volume of solvents and low yield of compound (I) thus the process is not economically viable. The reaction is represented by the scheme 2 as below:

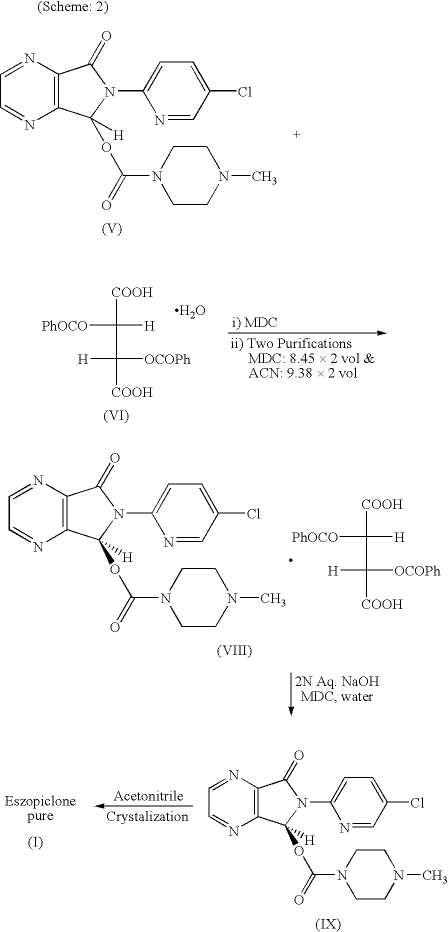

U.S. Pat. No. 6,864,257 (hereinafter referred as US '257) which is a divisional of U.S. Pat. No. 6,444,673, describes S(+) Zopiclone prepared from the corresponding racemate by methods such as chiral-phase chromatography, resolution of an optically active salt, stereoselective enzymatic catalysis by means of an appropriate microorganism or asymmetric synthesis. More especially the dextrorotatory isomer of Zopiclone is obtained by resolution of Zopiclone by means of an optically active acid like D(+)-O,O-dibenzoyl tartaric acid, in a yield of 23%. This invention also relates to pharmaceutical compositions containing dextrorotatory isomer of Zopiclone or its pharmaceutically acceptable salts. The patent US '257 also describes recovery of R-isomer of Zopiclone (example 1) from the mother liquor of tartarate salt of Zopiclone by concentrating to dryness under reduced pressure to get the salt of R-isomer which on alkalinization using sodium hydroxide gives levorotatory isomer which is recrystallized in acetonitrile (yield-13.9%).

ES2101653 discloses (+)-6-(5-chloropyrid-2-yl)-7-oxo-vinyloxycarbonyloxy-5,6-dihydro-pyrrolo[3,4-b]pyrazine and its use in a process for the preparation of (+)-6-(5-chloropyrid-2-yl)-5-(4-methylpiperazin-1-yl)-carbonyloxy-7-oxo-5,6-dihydropyrrole (3,4b) pyrazine. The synthesis of (+)-Zopiclone from (+)-6-(5-chloropyrid-2-yl)-7-oxo-5-vinyloxycarbonyloxy-5,6-dihydropyrrole (3,4b) pyrazine obtained by enzymatic resolution of its racemate from Candida antartica lipase as catalyst is disclosed in this reference.

U.S. Pat. No. 6,969,767 discloses process for the preparation of (S) Zopiclone by reacting N-methylpiperazine with optically enriched carbonate, namely, (S)-5-(chloromethyl oxy carbonyl oxy)-6-(5-chloropyridin-2-yl)-7-oxo-5,6-dihydro-pyrrolo[3,4-b]pyrazine. This process does not describe the recovery of R-isomer of Zopiclone.

WO/0069442 encompasses the synthesis, use and pharmaceutical compositions of (+)-N-desmethylzopiclone or optically pure enantiomers of N-desmethylzopiclone to treat or prevent diseases and conditions which are affected by the modulation of one or more central or peripheral benzodiazepine receptors. Optically pure (S) Zopiclone is treated with α-chloroethyl chloroformate to form the corresponding quaternary amine salt, which on methanolysis gives (S) desmethylzopiclone. Also disclosed is the preparation of optically pure (S) desmethylzopiclone using resolving agent such as L-N-benzyloxycarbonyl phenylalanine (L-ZPA). (+)-N-desmethylzopiclone and its (−) enantiomer in the mother liquor can be racemized under basic conditions (e.g. with a tertiary amine) to obtain racemic (+)-N-desmethylzopiclone. However since Zopiclone formed under these conditions is not stable, hence an alternative route for the recycling the enantiomer R-Zopiclone (X) is described as outlined in scheme 3.

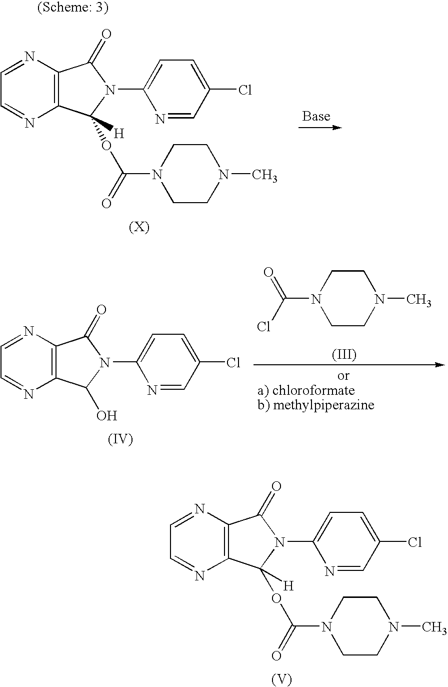

One more approach in same application involves dynamic resolution of 6-(5-chloropyrid-2-yl)-5-hydroxy-7-oxo-5,6-dihydropyrrolo[3,4-b]pyrazine (IV) with chiral auxiliary-based chloroformate to give one major stereoisomer of the carbonate product. This carbonate stereoisomer on reaction with N-methyl piperazine gives Eszopiclone (I) represented as below in scheme 4.

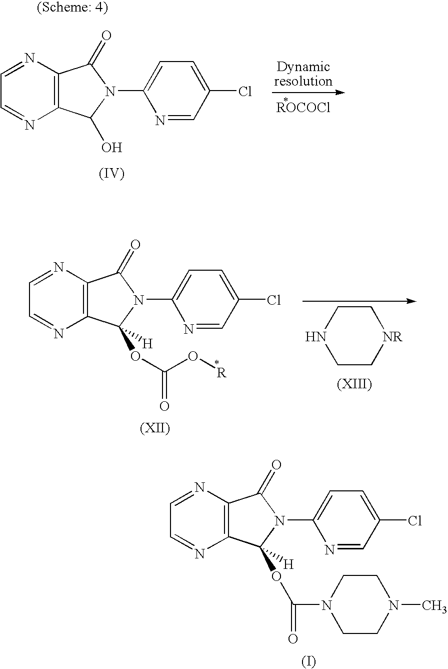

Recovery of starting material (IV) from unwanted isomer and thereby preparation of required enantiomer of Zopiclone is not mentioned clearly in WO00/69442 and hence, optimized process and yields are not available, calculated yields of all the steps are low and hence there is a need to develop cost effective as well as rugged process for preparation of Eszopiclone. Another approach in the same application is the synthesis of optically pure (+)-N-desmethylzopiclone by treating Zopiclone with diethyl azodicarboxylate and hydrolyzing the resulting product under mild conditions.

US2007054914 discloses process for the preparation of Eszopiclone by reacting Zopiclone with an enantiomerically pure di-p-toluoyl tartaric acid; and recovering solid salt by treatment with base to form Eszopiclone. This patent application also discloses reaction of 6-(5-chloropyrid-2-yl)-5-hydroxy-7-oxo-5,6-dihydropyrrolo[3,4-b]pyrazine with 1-chlorocarbonyl-4-methylpiperazine hydrochloride to form Zopiclone.

The process for synthesis of Eszopiclone as mentioned in prior art have the following drawbacks:

a) Use of equimolar amounts of sodium hydride and N-methyl piperazine carbonyl chloride as in U.S. Pat. No. 3,862,149, does not ensure completion of the reaction. Also racemic Zopiclone obtained is subjected to column chromatography, requires of large amounts of solvents like ethyl acetate (415 volumes) and methanol (13.3 volumes). The product is further recrystallized with a mixture of solvents such as acetonitrile and diisopropyl ether (7 volumes each). This method is unproductive, uneconomical and time consuming at industrial scale and results in low yield.

b) As per the process disclosed in U.S. Pat. No. 3,862,149, mode of addition of reagents, e.g. taking total amount of sodium hydride initially in the reaction vessel and adding piperazinyl acid chloride to it results in exotherm, which may lead to formation of side products thereby affecting the yield of desired compound. Method like column chromatography is required to purify the product making the process costly and industrially unsuitable.

c) WO00/69442 discloses recovery of R isomer of N-desmethylzopiclone from the mother liquor under basic conditions (e.g. tertiary amine) to obtain racemic Zopiclone. However racemic Zopiclone thus recovered from the undesired enantiomer is unstable and results in low yields.

d) As per the process for resolution in WO9212980 and U.S. Pat. No. 6,444,673, D(+)-tartarate salt of Zopiclone is prepared by dissolving Zopiclone and the chiral acid in dichloromethane (12.88 volumes) followed by evaporation of dichloromethane. Further to get pure tartarate salt, three crystallizations are required, one in 85 volumes of acetonitrile and rest of the two crystallizations are carried out in dichloromethane and acetonitrile mixture (8.45×2:9.38×2 volumes). Total amount of dichloromethane required is 29.78 vol. and acetonitrile required is 104.61 vol. Due to usage of high amounts of solvents, the batch size increases and process becomes less productive. The large amount of mother liquor containing mixture of solvents poses recovery problems and the process becomes uneconomical.

e) Recovery of 6-(5-chloropyrid-2-yl)-5-hydroxy-7-oxo-5,6-dihydropyrrolo[3,4-b]pyrazine (IV) from R-Zopiclone using a base is disclosed in WO00/69442 is not preferred due to lower yield of recovered compound (IV).

The process of the present invention differs from the prior art mentioned as in the above patents with respect to the following advantages:
1] Mole ratios of sodium hydride and N-methyl piperazine carbonyl chloride free base (III) with respect to key raw material compound (IV) is excess to ensure completion of the reaction.
2] Mode of addition of reagents avoids or minimize exotherms, so as to reduce side product formation/impurities thereby improving the yield of Eszopiclone. The O-acylation reaction is much simpler and chances of other side products are negligible.
3] Avoids multiple crystallization and column chromatography of racemic Zopiclone (V) as mentioned in prior art thereby reducing the process time and solvent consumption as well as energy cost required for solvent recovery.
4] Preparation of D(+)-O,O-dibenzoyl tartarate salt (VII).
5] Purification of D(+)-O,O-dibenzoyl tartarate salt (VII).
6] Conversion of R-isomer of Zopiclone using halogenated aliphatic acid to compound (IV) in higher yield.
7] Recovery of unwanted R-isomer of Zopiclone by simple method and reproducing Eszopiclone in high yield.

Therefore present invention addresses the problems associated with prior art and provides a process that is simple, efficient, inexpensive, ecofriendly, robust, readily scaleable, reproducible and commercially feasible. Hence the process of the invention produces the desired compound Eszopiclone (I) in high yield and purity. Further method of recovering key starting material of compound (IV) from the unwanted R-isomer of Zopiclone is provided herein.

### OBJECT OF THE INVENTION

The main object of the present invention is to provide process for the preparation of (S)-6-(5-chloro-pyrid-2-yl)-5-[(4-methyl-1-piperazinyl)carbonyloxy]-7-oxo-6,7-dihydro-5H-pyrrolo[3,4-b]pyrazine (I) in yield and high chemical and optical purity.

Another object of the invention is to provide recovery of key starting material 6-(5-chloro pyrid-2-yl)-5-hydroxy-7-oxo-5,6-dihydropyrrolo[3,4-b]pyrazine of formula (IV) from unwanted R-isomer and converting into racemic Zopiclone and finally to pure (S)(+) Zopiclone.

Another object of present invention is to provide cost effective, efficient, economical and industrially feasible process for the preparation of dextrorotatory isomer of Zopiclone (I) in high yield.

### SUMMARY OF THE INVENTION

The present invention discloses process for the preparation of (S)-6-(5-chloro-pyrid-2-yl)-5-[(4-methyl-1-piperazinyl)carbonyloxy]-7-oxo-6,7-dihydro-5H-pyrrolo[3,4-b]pyrazine (I) in high yield and high chemical and optical purity. Further disclosed herein is the recovery of key starting material of compound (IV) from unwanted R-isomer followed by conversion to compound of formula (I) in higher yield.

The process for the preparation of (S)(+) Zopiclone according to the present invention, comprises the steps of;
a) basifying N-methyl piperazine carbonyl chloride hydrochloride (II) by using base to obtain N-methyl piperazine carbonyl chloride as a free base of formula (III);
b) condensing N-methyl piperazine carbonyl chloride free base (III) with 6-(5-chloro pyrid-2-yl)-5-hydroxy-7-oxo-5,6-dihydropyrrolo[3,4-b]pyrazine (IV) in presence of molar excess of base in a dipolar aprotic solvent to obtain racemic Zopiclone (V);
c) reacting compound (V) with D(+)-O,O′-dibenzoyl tartaric acid monohydrate (VI) in acetonitrile and filtering the precipitated solid to isolate D(+)-O,O′-dibenzoyl tartarate salt (VII);
d) purifying D(+)-O,O′-dibenzoyl tartaric acid salt (VII) to obtain pure compound (VIII);
e) breaking the tartarate salt of esopiclone (VIII) using an aqueous solution of base to get crude eszopiclone (IX);
f) purifying crude eszopiclone (IX) in acetonitrile to obtain pure eszopiclone (I) substantially free of its R-isomer,
g) optionally isolating the R-isomer of Zopiclone by treating the filtrates obtained from step (c) and (d);

The unwanted R-isomer present in the filtrates obtained from the preparation and purification of enantiomerically enriched D(+)-O,O-dibenzoyl tartarate salt (VII), is isolated by evaporating the solvent to yield a residue, which on basification with 2N aqueous NaOH in presence of dichloromethane provides the R-Zopiclone (X).

According to another aspect of the present invention, there is provided a process for conversion of unwanted R-isomer of Zopiclone to compound (IV) which comprises:
a) evaporating the filtrates collected from step c) and step d) to obtain residue;
b) treating the obtained residue with base and isolating R-zopiclone (X);
c) cleaving the R-Zopiclone (X) with acid in organic solvent or water or mixtures thereof;
d) neutralizing the obtained reaction mixture with base to precipitate out compound (IV);
e) isolating the compound (IV) by filtration followed by washing with water and then with organic solvent.

The isolated compound (IV) is obtained in high yield and purity which is then converted to (S)(+) Zopiclone (I) by known methods.

According to another aspect of the present invention, there is provided a process for preparation of pure Eszopiclone from unwanted R-Zopiclone which comprises the steps of:
a) treating the R-Zopiclone (X) with acid in organic solvent or water or mixtures thereof;
b) neutralizing the obtained reaction mixture with base to precipitate out compound (IV);
c) condensing compound (IV) with molar excess of N-methyl piperazine carbonyl chloride (III) in presence of molar excess of base in a dipolar aprotic solvent to obtain racemic zopiclone (V);
d) reacting compound (V) with D(+)-O,O′-dibenzoyl tartaric acid monohydrate (VI) in acetonitrile and filtering the precipitated solid to isolate enantiomerically enriched D(+)-O,O′-dibenzoyl tartarate salt of zopiclone (VII);
e) purifying D(+)-O,O′-dibenzoyl tartaric acid salt of Zopiclone (VII) to get the pure tartarate salt of eszopiclone (VIII);
f) breaking the tartarate salt of eszopiclone (VIII) using base to get crude Eszopiclone (IX); and
g) purifying crude Eszopiclone (IX) to obtain pure Eszopiclone (I) substantially free of R-isomer (X).

The present invention thus provides an efficient synthetic process for the preparation of (S)(+) Zopiclone in high yield and purity and simple recovery and recycling of the unwanted R-isomer which is further converted into Eszopiclone (I).

### BRIEF DESCRIPTION OF FIGURES

FIG. 1 is a characteristic X-ray Powder diffraction pattern of Eszopiclone prepared according to the present invention.

### DETAILED DESCRIPTION OF THE INVENTION

The present invention describes an efficient process for the preparation of (S) (+) 6-(5-chloro-pyrid-2-yl)-5-[(4-methyl-1-piperazinyl)carbonyloxy]-7-oxo-6,7-dihydro-5H-pyrrolo[3,4-b]pyrazine (I) with in yield and high chemical and optical purity. The invention further describes recovery of key starting material of compound (IV) from the unwanted R-isomer of Zopiclone, converting into racemic Zopiclone and finally to Eszopiclone.

The process sequence of the present invention is represented by the scheme 5 as below:

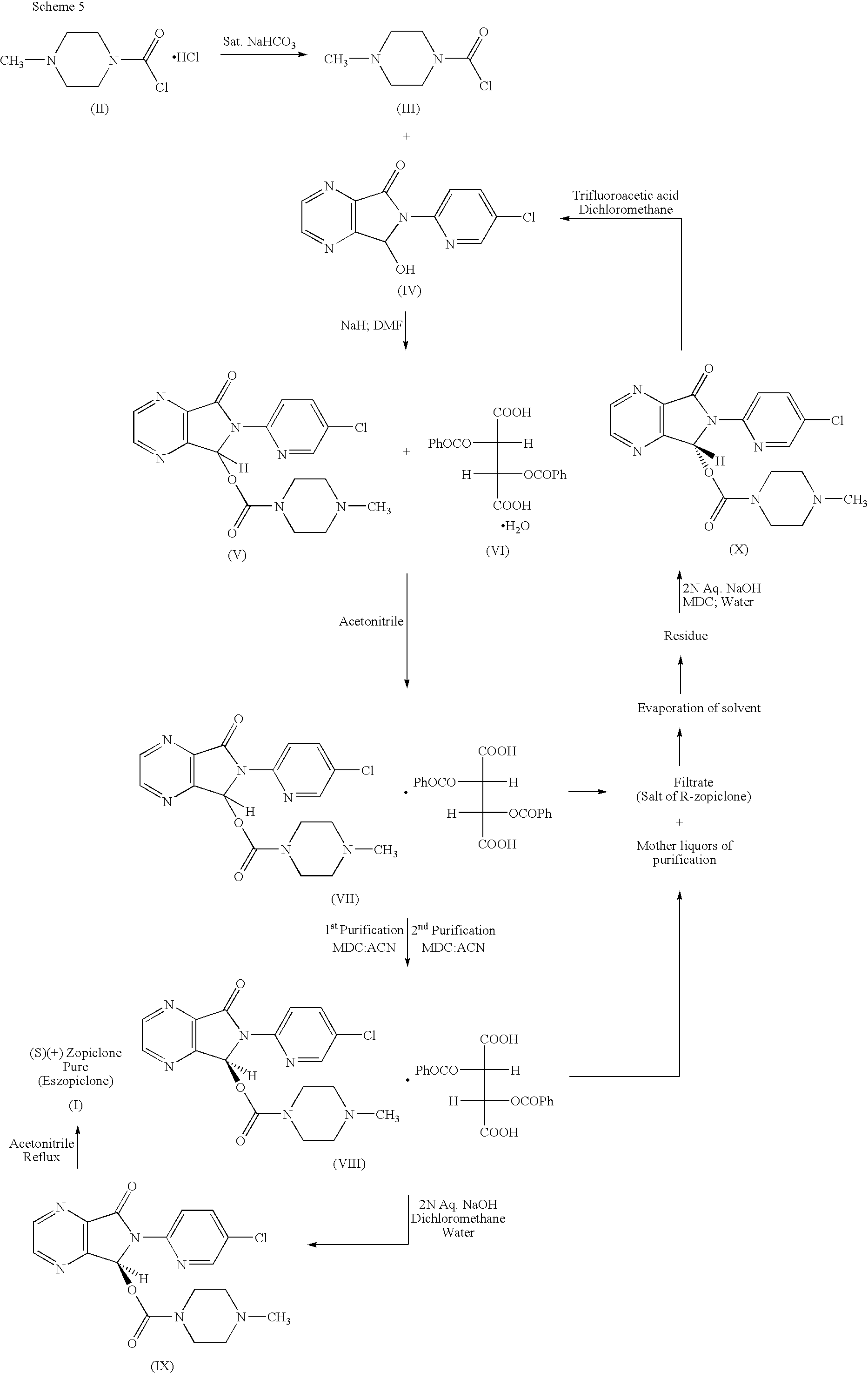

A process for the preparation of (S)(+) Zopiclone according to the present invention, comprises the steps of:
a) basifying N-methyl piperazine carbonyl chloride hydrochloride (II) by using base to obtain N-methyl piperazine carbonyl chloride (III);
b) condensing N-methyl piperazine carbonyl chloride (III) with molar excess of 6-(5-chloropyrid-2-yl)-5-hydroxy-7-oxo-5,6-dihydropyrrolo[3,4-b]pyrazine (IV) in presence of molar excess of base in a dipolar aprotic solvent to obtain racemic Zopiclone (V);
c) reacting compound (V) with D(+)-O,O′-dibenzoyl tartaric acid monohydrate (VI) in acetonitrile and filtering the precipitated solid to isolate D(+)-O,O′-dibenzoyl tartarate salt of Zopiclone (VII);
d) purifying D(+)-O,O′-dibenzoyl tartaric acid salt (VII);
e) breaking the tartarate salt of Zopiclone (VII) using base to dextrorotatory isomer of Zopiclone i.e. crude Eszopiclone (IX);
f) purifying crude Eszopiclone (IX) in acetonitrile to obtain pure Eszopiclone (I) substantially free of R-isomer,
g) optionally isolating the R-isomer of Zopiclone from the mother liquor obtained from step (c) and (d);

The first step of the process for preparation of Eszopiclone (I) involves neutralizing compound (II) in organic solvent using aqueous solution of base till the pH of the reaction mixture is alkaline and isolating the compound (III).

In preferred embodiment, N-methyl piperazine carbonyl chloride hydrochloride (II) is mixed with organic solvent. Further to the resulting suspension, an aqueous saturated solution of base is slowly added under stirring. During basification the N-methyl-piperazine carbonyl chloride gets extracted in organic solvent layer, which is separated from the aqueous layer. The aqueous layer is further extracted with organic solvent. The combined organic layers are washed with water, dried over anhydrous sodium sulfate and concentrated under vacuum to isolate N-methyl piperazine carbonyl chloride (III).

The neutralization is carried out at 0 to 20° C. preferably 0 to 5° C. The alkaline pH of reaction mixture is in the range of 7.5 to 9.0, preferably 8.0.

The organic solvent used is selected from the group consisting of halogenated hydrocarbons such as dichloromethane, chloroform, or esters such as ethyl acetate or ethers such as diethyl ether, methyl t-butyl ether or mixtures thereof, preferably dichloromethane.

The base used is selected from the group consisting of alkali metal or alkaline earth metal carbonates or bicarbonates preferably bicarbonates and more preferably sodium bicarbonate.

The second step of the present invention involves addition of base in portions to the solution of 6-(5-chloro pyrid-2-yl)-5-hydroxy-7-oxo-5,6-dihydropyrrolo[3,4-b]pyrazine (IV) in dipolar aprotic solvent to obtain a reaction mixture. The addition is preferably carried out under cold conditions. The ratio of base to compound (IV) used is in molar excess. Solution of N-methyl piperazine carbonyl chloride (NMPCCl) free base (III) in dipolar aprotic solvent is then slowly added with the stirring to the above reaction mixture. The addition is preferably carried out under cold conditions. The additions are carried out at the lower temperature range of −5 to −15° C., preferably at −10° C. The mixture is stirred at temperature below 20° C., preferably 15 to 18° C. for 2 to 4 hours. After completion of reaction, the reaction mixture is quenched in 3 to 10 volumes, preferably 4.5 volumes (with respect to reaction medium) of ice-water and stirred for 15 to 45 minutes, preferably 20 minutes during which the product gets separated, which is then isolated by filtration, washed with water followed by ether and dried to get racemic zopiclone (V). The racemic zopiclone thus obtained is further resolved to get its S-isomer.

The dipolar aprotic solvent is selected from anhydrous N,N-dimethylacetamide, N,N-dimethylformamide, N-methyl-2-pyrrolidone or mixture thereof, preferably N,N-dimethylformamide.

The base is alkali metal hydride such as sodium hydride (50%-60% suspension in mineral oil). Sodium hydride and NMPCCl free base (III) are used in molar excess with respect to 6-(5-chloropyrid-2-yl)-5-hydroxy-7-oxo-5,6-dihydropyrrolo[3,4-b]pyrazine of formula (IV). Preferably the molar ratio of compound (IV):sodium hydride:NMPCCl free base (III) is 1:1.3:1.4.

The third step of the present invention involves resolution of racemic Zopiclone (V) using D(+)-O,O′-dibenzoyl tartaric acid monohydrate (VI) to isolate optically enriched Eszopiclone. The resolution of Zopiclone involves formation of tartarate salt by addition of chiral acid to solution of racemic Zopiclone (V) in acetonitrile. The solution is stirred at room temperature to precipitate out the chiral acid salt (VII). The precipitated crude enantiomerically enriched chiral salt of Zopiclone (VII) is isolated by filtration. The filtrate enriched in R-isomer is used for recovery of compound (IV).

The chiral acid is selected from D-Tartaric acid, D(+)-O,O′-dibenzoyl tartaric acid monohydrate, (+)-O,O′-Di-p-toluoyl tartaric acid, preferably D(+)-O,O′-dibenzoyl tartaric acid monohydrate (VI).

The amount of acetonitrile used is 20 to 80 volumes, preferably 30 volumes with respect to compound of formula (V). The amount of compound of formula (VI) is 0.5 to 1.1 molar in relation to compound of formula (V).

The fourth step of the present invention involves purification of enantiomerically enriched D(+)-O,O′-dibenzoyl tartaric acid salt (VII) for isolating pure D(+)-O,O′-dibenzoyl tartaric acid salt of (S)(+)-Zopiclone (VIII). Accordingly, the crude salt (VII) is dissolved in organic solvent under reflux condition. The obtained reaction mixture is filtered. To this filtrate under hot condition, an anti-solvent is added slowly, while the temperature of the mixture is gradually raised. After addition of anti solvent, the reaction mixture is heated followed by stirring at temperature of 50 to 65° C. for 5 to 25 minutes, preferably at 55° C. for 15 minutes. Thereafter, the mixture is allowed to cool to a temperature of 25 to 30° C. and maintained at temperature of 2 to 10° C., preferably 5° C. for one hour during which the product gets separated, which is then isolated. The same purification process may be repeated to get purified tartarate salt of Eszopiclone (VIII) free from unwanted R-enantiomer. The filtrate is set aside for recovery of R-isomer and its further conversion to Eszopiclone (I).

The organic solvent is selected from dichloromethane, chloroform or mixture thereof, preferably dichloromethane and used in 15 to 30 volumes, preferably 20 volumes with respect to compound of formula (VII). The anti-solvent used is acetonitrile and used in 20 to 40 volumes, preferably 25 volume with respect to compound (VII).

In the fifth step of the present invention, dextrorotatory isomer of Zopiclone is isolated by breaking of the tartarate salt (VIII) by using aqueous solution of base in molar excess. Preferably, purified salt of Eszopiclone (VIII) is stirred with organic solvent and water at room temperature at 25 to 35° C. for 5 to 15 minutes, preferably 10 minutes followed by alkalinizing the mixture by slow addition of aqueous solution of base.

The organic layer is separated and aqueous layer is further extracted thrice with the organic solvent. The combined organic layers are washed with water, dried over anhydrous sodium sulphate and solvent is evaporated under reduced pressure to get Eszopiclone crude (IX).

The base used is alkali metal hydroxide selected from sodium hydroxide, potassium hydroxide, preferably sodium hydroxide. The organic solvent is selected from dichloromethane, chloroform or mixture thereof, preferably dichloromethane.

The volume of dichloromethane and water used for extraction is 6 to 10 volumes each, preferably 7.6 volumes. The molar excess of sodium hydroxide used is 1.5 to 2.5 equivalents, preferably 2.1 equivalents with respect to compound (VIII).

In the sixth step of the invention, the isolated Eszopiclone (IX) is further purified to get ICH passing material along with higher optical purity which involves crystallization of compound (IX) in acetonitrile. The crystallization process involves dissolution of crude Eszopiclone (IX) in acetonitrile under reflux condition for 5 to 15 minutes to get a clear solution. Activated charcoal (5% w/w) is added to clear solution of Eszopiclone followed by filtration of the mixture through hyflo bed. The filtered solution is stirred and allowed to reach room temperature and then cooled at temperature of 5 to 15° C. for 15 to 60 minutes. The crystalline solid obtained is then filtered, washed with cold acetonitrile and dried with suction at temperature range of 55 to 65° C. for 4 hours to get highly pure Eszopiclone (I). Acetonitrile used for purification of Eszopiclone (IX) is preferably 9.5 to 12 volumes, more preferably 10 volumes to get highly pure Eszopiclone (I).

Eszopiclone obtained according to the present invention is highly pure with unwanted (R)-isomer (X) and N-desmethylzopiclone (XI) being less than 0.1% and with chemical purity of more than 99.5% by HPLC.

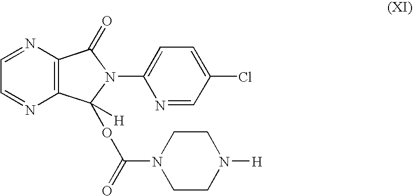

The term “chemical purity” as described herein refers to purity of Eszopiclone which is more than 99.5%.

The term “enantiomerically enriched” used herein refers to mixtures of the particular (R) and (S) enantiomers in which one enantiomer is present in an enantiomeric excess in comparison to the other enantiomer.

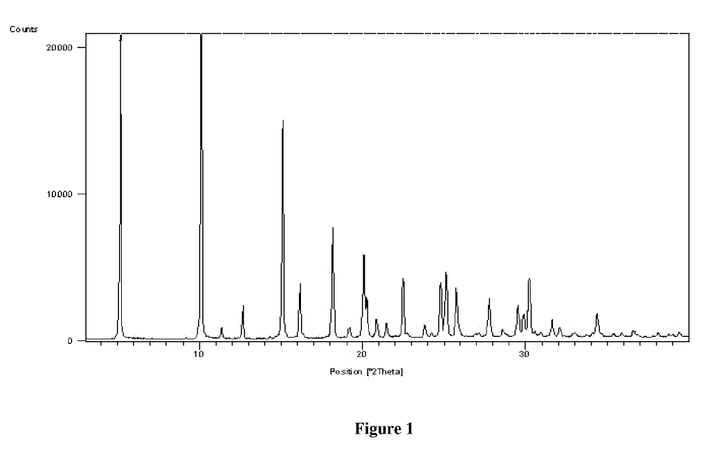

Eszopiclone obtained by the process of the present invention is characterized by the X-ray powder diffraction pattern [as shown in FIG. 1] having peaks at about 2θ: 5.16; 9.15; 10.11; 11.35; 12.66; 14.29; 15.09; 16.15; 18.16; 19.14; 20.09; 20.85; 21.46; 22.01; 22.50; 23.83; 24.24; 24.79; 25.13; 25.76; 26.89; 27.14; 27.79; 28.58; 29.49; 29.88; 30.21; 30.57; 30.91; 31.60; 32.07; 33.02; 33.66; 34.07; 34.37; 35.34; 35.85; 36.59; 38.10; 38.75; 39.36±0.2 degrees.

Alternatively crude Eszopiclone is purified by dissolving crude Eszopiclone in an organic solvent and isolating pure Eszopiclone (I). Optionally the pure Eszopiclone (I) is isolated by addition of an antisolvent.

The organic solvent used is an ester, preferably ethyl acetate and antisolvent used is an aliphatic or aromatic hydrocarbon preferably hexane.

Another embodiment of the invention encompasses method of recovery of 6-(5-chloropyrid-2-yl)-5-hydroxy-7-oxo-5,6-dihydropyrrolo[3,4-b]pyrazine (IV) from the unwanted R-isomer (X), which is an inventive synthetic process.

Recovery of 6-(5-chloro pyrid-2-yl)-5-hydroxy-7-oxo-5,6-dihydropyrrolo[3,4-b]pyrazine (IV) from unwanted (R)-Zopiclone (X) involves combining all the mother liquors enriched in (R)-Zopiclone D(+)-O,O′-dibenzoyl tartarate salt and isolating the crude solid by evaporating the solvent.

In a preferred embodiment, breaking of the tartarate salt of (R)-Zopiclone by using aqueous solution of inorganic base to get (R)-Zopiclone (X) involves stirring the tartarate salt of (R)-Zopiclone with organic solvent and water at room temperature. The mixture is stirred at temperature of 25 to 35° C. for 5 to 15 minutes, preferably 10 minutes. Addition of the 2N solution of base is continued till alkaline pH, preferably pH 11. During addition, the temperature is maintained at 0 to 15° C. for 5 to 15 minutes, preferably at 5° C. for 5 minutes.

The reaction mixture is transferred to a separator. The organic layer is separated and aqueous layer is further extracted thrice with the organic solvent. The combined organic layer is washed with water, dried over anhydrous sodium sulphate and the solvent evaporated under reduced pressure to get (R)-Zopiclone (X).

The base used is selected from sodium hydroxide, potassium hydroxide, preferably sodium hydroxide. The molar excess of sodium hydroxide is 1.5 to 2.5 equivalents, preferably 2.1 equivalents.

The organic solvent is selected from dichloromethane, chloroform, or mixtures thereof, preferably dichloromethane.

The volumes of dichloromethane and water are 6 to 10 volumes, preferably 7.6 volumes each.

In a further embodiment (R)-Zopiclone is cleaved to obtain compound (IV) comprising:
a) cleaving the R-Zopiclone (X) with acid in organic solvent or water or mixtures thereof;
b) neutralizing the obtained reaction mixture with the base to precipitate out compound (IV); and
c) isolating the compound (IV) by filtration followed by washing with water and then with organic solvent.

Preferably, (R)-Zopiclone is cleaved in the presence of acid in organic solvent under reflux condition for 10 to 30 hours, preferably at temperature of 38° C. for 22 hours and then quenched in ice-water mixture. The pH of the reaction mixture is made slightly alkaline by addition of aqueous saturated solution of base till pH between 7 to 11, preferably 7.5 to 8 is achieved at temperature 0 to 15° C., preferably at 5° C. to precipitate out compound, 6-(5-chloropyrid-2-yl)-5-hydroxy-7-oxo-5,6-dihydropyrrolo[3,4-b]pyrazine (IV) which is filtered, washed with water and then with organic solvent and dried at temperature of 55 to 65° C. for 2 to 20 hours, preferably at 60° C. for 10 hours.

The compound (IV) thus recovered is in high yield as compared to methods disclosed in the prior art, thereby making the process economically viable.

In another embodiment, (R)-Zopiclone (X) is stirred with a mixture of acid, water and alcohol at temperature 41° C. for 11 hours. The obtained reaction mixture is cooled to 10 to 15° C. and then neutralized with saturated solution of sodium bicarbonate. Solid precipitated out is filtered, washed with water and then with organic solvent to get compound (IV) in high yield.

The acid used for cleavage of the R-isomer is selected from trifluoroacetic acid, trichloroacetic acid, preferably trifluoroacetic acid and the inorganic acid is hydrochloric acid.

Organic solvent is selected from dichloromethane, chloroform, ethylene dichloride, 1,1,1-trichloroethane, chlorobenzene or mixture thereof, preferably dichloromethane and the alcohol is selected from methanol, ethanol, isopropanol preferably methanol.

The base used is selected from the group consisting carbonate or bicarbonate of alkali metal or alkaline earth metal, more preferably sodium bicarbonate.

In another aspect of the invention the recovered 6-(5-chloropyrid-2-yl)-5-hydroxy-7-oxo-5,6-dihydropyrrolo[3,4-b]pyrazine (IV) is converted to Eszopiclone of desired optical and chemical purity by the process as described in the current invention or by known methods.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention.

### Example: 1

### Conversion of N-Methyl Piperazine Carbonyl Chloride Hydrochloride Salt (II) to N-Methyl Piperazine Carbonyl Chloride Base (III)

250 g (1.25 M) N-methyl-piperazine carbonyl chloride hydrochloride was stirred in 1574 ml of dichloromethane while the temperature being maintained at 5 to 10° C. The mixture was then neutralized by slow addition of 1574 ml of saturated solution of sodium bicarbonate till pH of 7.5 to 8.0 was achieved. During addition, the temperature was maintained at 5° C. After complete addition of sodium bicarbonate, reaction mixture was stirred for 30 minutes and then transferred to a separator. Bottom layer of dichloromethane was separated and the aqueous layer extracted with 2×800 ml of dichloromethane. Dichloromethane layers were combined and washed with 1000 ml of water, dried over anhydrous sodium sulphate and evaporated under reduced pressure to get N-methyl piperazine carbonyl chloride base (III). Yield: 133 g, 65.14%.

### Example 2

### Synthesis of Racemic Zopiclone (V)

150 g (0.57 M) of 6-(5-chloropyrid-2-yl)-5-hydroxy-7-oxo-5,6-dihydropyrrolo[3,4-b]pyrazine (IV) was dissolved in 4.5 liter (30 volumes) of anhydrous N,N-dimethylformamide at 25 to 35° C. and mixture was stirred for 20 minutes. The solution was then cooled and 29.62 gm (0.74 M) of sodium hydride (50 to 60% suspension in mineral oil) was added in portions to the cooled solution with stirring. During addition, the temperature was maintained at −10° C. After complete addition, the reaction mixture was stirred at same temperature for evolution of H₂ gas. A solution of 131 gm (0.8 M) of N-methyl piperazine carbonyl chloride (NMPCCl base) (III) in anhydrous N,N-dimethylformamide was slowly added. The addition was carried out at temperature of −10° C. After complete addition of NMPCCl base, the temperature was allowed to rise gradually. The mixture was stirred at temperature below 20° C. for three hours. The mixture was then quenched in 18.6 kg of ice-water and stirred for 20 minutes. Solid precipitated out was filtered off, washed with 2 liter of water, then with 1250 ml of diisopropyl ether. The product was dried at temperature of 65° C. for 18 hrs. Yield: 175 g, 78.81%.

### Example 3

### Preparation of D(+)-O,O-dibenzoyl Tartarate of Zopiclone (VII)

175 g (0.45 M) of racemic Zopiclone (V) was dissolved in 5250 ml of acetonitrile at temperature of 25 to 35° C. with stirring. 169.37 g (0.45 M) of D(+)-O,O′-dibenzoyl tartaric acid monohydrate was then added to the above solution of Zopiclone with stirring. The reaction mixture was stirred at temperature of 25 to 35° C. for 6 to 7 hrs. The solid precipitated out was filtered off and washed with 2×200 ml of acetonitrile. The crude D(+)-O,O′-dibenzoyl tartarate salt of Zopiclone thus obtained was dried at 60 to 65° C. for 10 hours. Yield: 298 g, 86.63%.

### Example 4

### Purification of D(+)-O,O′-tartarate Salt of Zopiclone (VII)

The D(+)-O,O′-dibenzoyl tartarate of zopiclone was subjected to two consecutive purifications as follows.

A] 298 g of D(+)-O,O′-dibenzoyl tartarate of Zopiclone (VII) was dissolved in 5960 ml of dichloromethane under reflux. The mixture was filtered to remove any inorganic matter. The filtrate was then refluxed for 5 minutes and to it 7301 ml of acetonitrile was slowly added. Reaction mixture was maintained at temperature of 50 to 55° C. for 15 minutes and then allowed to reach room temperature. The mixture was then maintained at 5° C. for 1 hour. The crystallized product was filtered off, washed with 2×450 ml of acetonitrile and suck dried.

B] The crystallized D(+)-O,O′-dibenzoyl tartaric acid salt of Eszopiclone thus obtained was crystallized again in the same solvents as in step A above, however the solution of salt in dichloromethane was not filtered at this stage. Purified D(+)-O,O′-dibenzoyl tartaric acid salt of Eszopiclone obtained has a m.p. of 160 to 165° C. (with decomposition), the optical rotation [α]₂₀=102°±3° (c=0.5; acetone). Yield: 154.47 g, 44.85%.

### Example 5

### Isolation of Crude Eszopiclone (IX) from Purified D(+)-O,O-dibenzoyl Tartarate Salt of (S)-Zopiclone (VIII)

154.47 g (0.2 M) of purified D(+)-O,O-dibenzoyl tartarate salt of Eszopiclone (VIII) was dissolved in 1174 ml of water in the presence of 1174 ml of dichloromethane. The mixture was alkalinized to attain pH 11 by slowly adding 2N aqueous sodium hydroxide solution. The aqueous phase was separated from the organic phase and the aqueous phase was further extracted with 2×500 ml of dichloromethane. The combined organic phases were washed with 3×1000 ml of water and dried over sodium sulphate, evaporated to get crude Eszopiclone (IX). m.p.: 200±5° C., optical rotation [α]₂₀=135±3° (c=1.0; acetone). Yield: 69.62 g, 44.34%

### Example 6

69 g of Eszopiclone (IX) was dissolved in 696 ml of acetonitrile under reflux and 3.48 g of activated charcoal was added to the obtained solution with stirring. The hot solution was filtered through hyflo bed and the filtrate stirred at 10° C. The crystalline solid obtained was then filtered, washed with cold acetonitrile and dried at temperature of 55 to 65° C. for 4 hours. The pure dextrorotatory Eszopiclone (I) was obtained as colorless crystals, m.p. 206±5° C., optical rotation [α]₂₀=135±3° (c=1.0; acetone). Yield: 60.44 g, 34.53%.

### Example 7

6.0 g of Eszopiclone was dissolved in 174 ml of ethyl acetate under reflux and 0.3 g of activated charcoal was added to the obtained solution with stirring. The hot solution was filtered through hyflo bed and the filtrate was stirred at room temperature. The crystalline solid obtained was then filtered, washed with ethyl acetate and dried at temperature of 70 to 75° C. for 8 hours. The pure dextrorotatory Eszopiclone was obtained as colorless crystals, m.p. 206±5° C., optical rotation [α]₂₀=135±3° (c=1.0; acetone).

Yield: 4.9 g

### Example 8

6.9 g of crude eszopiclone was dissolved in 200 ml of ethyl acetate under reflux and 0.34 g of activated charcoal was added to the obtained solution with stirring. The hot solution was filtered through hyflo bed and the filtrate was stirred at room temperature. The crystalline solid obtained was then filtered, washed with ethyl acetate and dried at temperature of 70 to 75° C. for 8 hours. The pure dextrorotatory Eszopiclone (I) was obtained as colorless crystals, m.p. 206±5° C., optical rotation [α]₂₀=135±3° (c=1.0; acetone). Yield: 5.7 g

### Example 9

3.0 g of crude eszopiclone was dissolved in 87 ml of ethyl acetate under reflux and 0.14 g of activated charcoal was added to the obtained solution with stirring. The hot solution was filtered through hyflo bed and the filtrate was stirred at room temperature. 15 ml hexane was added to the obtained solution and stirred. The crystalline solid obtained was then filtered, washed with ethyl acetate and dried at temperature of 70 to 75° C. for 8 hours. The pure dextrorotatory Eszopiclone (I) was obtained as colorless crystals, m.p. 206±5° C., optical rotation [α]₂₀=135±3° (c=1.0; acetone). Yield: 2.65 g

### Example 10

### Isolation of R(−) Zopiclone

191.47 g (0.25 M) of D-O,O′-dibenzoyl tartarate salt of the levorotatory isomer of Zopiclone (obtained from the filtrates of the tartarate salt of (S)(+) isomer of Example 3 and 4) was dissolved in 1456 ml of water in the presence of 1456 ml of dichloromethane. The mixture was alkalinized to attain pH of 11 by slowly adding 2N aqueous sodium hydroxide solution. The aqueous phase was separated and extracted with dichloromethane (2×700 ml). The combined organic phases were washed with 3×1200 ml of water, dried over sodium sulphate and evaporated to obtain R-(−)Zopiclone (X). m.p. 185° C., optical rotation [α]₂₀=133±3° (c=1.0; acetone). Yield: 82 g, 46.85%.

### Example 11

### Recovery of 6-(5-chloro pyrid-2-yl)-5-hydroxy-7-oxo-5,6-dihydropyrrolo[3,4-b]pyrazine (IV) from Unwanted (R)-Zopiclone (X)

82 g (0.21 M) of (R)-Zopiclone (X) was dissolved in 820 ml of dichloromethane and to it was slowly added 410 ml (629.35 gm; 5.51 M) of trifluoroacetic acid with stirring. The mixture was stirred under reflux condition for 18 hours. The mixture was quenched in 1.65 kg of ice-water and to it sat. solution of sodium bicarbonate was added dropwise till pH of 7.5 to 8.0 was achieved. Solid obtained was washed with 400 ml of water and with 123 ml dichloromethane and dried. m.p. 240° C., optical rotation [α]₂₀=0.05 (c=1.0; dioxane). Yield: 38.7 g, 70%.

### Example 12

### Recovery of 6-(5-chloropyrid-2-yl)-5-hydroxy-7-oxo-5,6-dihydropyrrolo[3,4-b]pyrazine (IV) from Unwanted (R)-Zopiclone (X)

5.0 g (0.012 M) of (R)-Zopiclone (X) was stirred with a mixture of 12.84 ml of concentrated HCl, 12.84 ml of water and 5.0 ml of methanol at temperature 41° C. for 11 hours. The obtained reaction mixture was cooled to 10 to 15° C. and then neutralized with saturated solution of sodium bicarbonate. The solid precipitated out was filtered, washed with 30.0 ml water, then with 7.5 ml dichloromethane and dried. Yield; 1.39 g, 41.24%.

### Example 13

### Synthesis of Eszopiclone from the Recovered Compound (IV)

38.7 g (0.14 M) of the recovered 6-(5-chloropyrid-2-yl)-5-hydroxy-7-oxo-5,6-dihydropyrrolo[3,4-b]pyrazine (IV) was reacted with 33.79 g (0.2 M) of N-methyl piperazine carbonyl chloride base (III) in presence of 7.64 g (0.19 M) of sodium hydride (under same conditions as that of Example 2) to yield racemic Zopiclone. Racemic Zopiclone thus obtained was further converted to Eszopiclone (I) as in the foregoing examples.

1. A process for the preparation of optically pure Eszopiclone of formula (I) comprising the steps of: 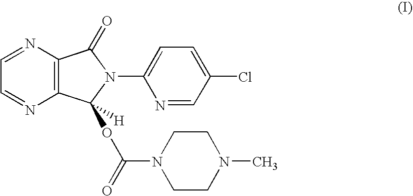
a) basifying N-methyl piperazine carbonyl chloride hydrochloride (11) using base to obtain N-methyl piperazine carbonyl chloride of formula (III); 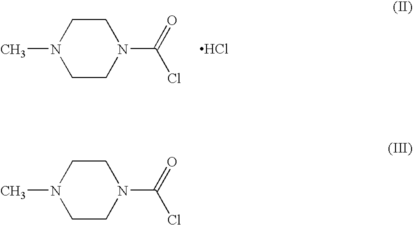
b) condensing 6-(5-chloropyrid-2-yl)-5-hydroxy-7-oxo-5,6-dihydropyrrolo[3,4-b]pyrazine (IV) with molar excess of N-methyl piperazine carbonyl chloride (III) in presence of molar excess of base in a dipolar aprotic solvent to obtain racemic zopiclone (V); 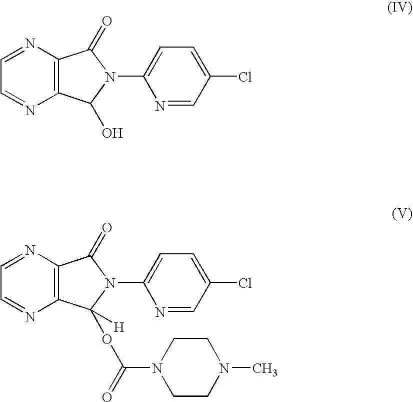
c) reacting compound (V) with D(+)-O,O′-dibenzoyl tartaric acid monohydrate (VI) in acetonitrile and filtering the precipitated solid to isolate enantiomerically enriched D(+)-O,O′-dibenzoyl tartarate salt of zopiclone (VII); 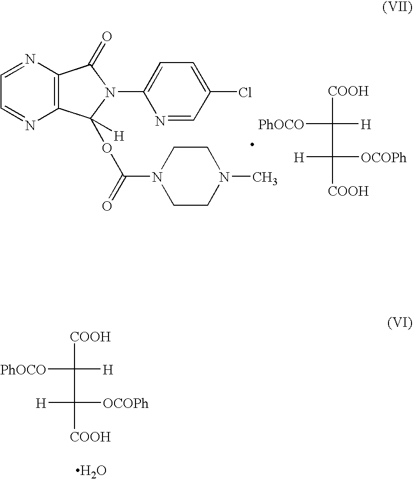
d) purifying D(+)-O,O′-dibenzoyl tartaric acid salt of zopiclone (VII) to get pure tartarate salt of eszopiclone (VIII); 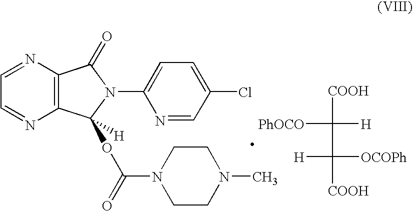
e) breaking the tartarate salt of (VIII) using an aqueous solution of base to get Eszopiclone (IX); 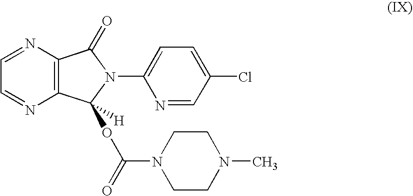
f) purifying crude Eszopiclone (IX) in acetonitrile to obtain pure Eszopiclone (I) substantially free of R-isomer (X); 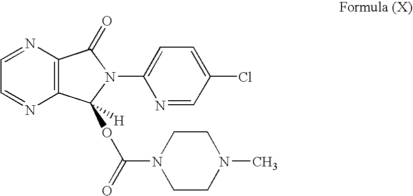
g) optionally isolating the R-isomer of Zopiclone from the filtrates obtained from step (c) and (d) and converting into Eszopiclone (I).

2. The process as claimed in claim 1, wherein compound (III) is liberated by neutralizing compound (II) in organic solvent selected from halogenated hydrocarbons, esters, ethers or mixtures thereof in presence of aqueous solution of base selected from alkali metal carbonates or bicarbonates.

3. The process as claimed in claim 2, wherein the reaction is carried out at low temperature below 25° C. for a period of 5 to 60 minutes till the pH of the reaction mixture is in the range of 7.5 to 9.

4. The process as claimed in claim 1, wherein the base used in step (b) is an alkali metal hydride.

5. The process as claimed in claim 1 wherein the dipolar aprotic solvent in step (b) is selected from N,N-dimethylformamide, N,N-dimethylacetamide, N-methylpyrrolidone or mixtures thereof.

6. The process as claimed in claim 1, wherein reaction of step (b) is carried out at a low temperature in the range of −15 to 0° C.

7. The process as claimed in claim 1 wherein enantiomerically enriched tartarate salt of zopiclone (VII) in step (c) is obtained by;
a) dissolving the racemic Zopiclone (V) in acetonitrile;
b) adding D(+)-O,O′-dibenzoyl tartaric acid monohydrate (VI) to the obtained solution and stirring for 4 to 24 hours at temperature range of 25 to 35° C. to precipitate out the solid;
c) isolating compound (VII) by filtration.

8. The process as claimed in claim 7, wherein acetonitrile is used in an amount of 20 to 80 volumes.

9. The process as claimed in claim 1, wherein the purification of enantiomerically enriched D(+)-O,O′-dibenzoyl tartaric acid salt of zopiclone (VII) in step d) comprises;
a) refluxing compound of formula (VII) in organic solvent selected from dichloromethane, chloroform or mixtures thereof;
b) stirring the reaction mixture followed by addition of acetonitrile;
c) heating the reaction mixture at temperature of 50 to 65° C. for few minutes and
d) isolating pure compound (VIII).

10. The process as claimed in claim 9, wherein the amount of organic solvent used is 15 to 30 volumes.

11. The process as claimed in claim 9, wherein the amount of acetonitrile used is 20 to 40 volumes.

12. The process as claimed in claim 1, wherein the base used in step (e) is alkali metal hydroxide selected from sodium hydroxide or potassium hydroxide.

13. The process as claimed in claim 1, wherein isolation of (R)-Zopiclone and its conversion to Eszopiclone comprises the steps of;
a) evaporating the filtrates collected from step c) and step d) to obtain residue;
b) treating the obtained residue with base and isolating R-zopiclone (X);
c) cleaving the R-Zopiclone (X) with acid in organic solvent or water or mixtures thereof;
d) neutralizing the obtained reaction mixture with base to precipitate out compound (IV);
e) isolating the compound (IV) by filtration followed by washing with water and then with organic solvent and
f) converting the isolated compound (IV) into Eszopiclone (I) by known methods.

14. A process for preparation of Eszopiclone comprising the steps of;
a) treating the R-Zopiclone (X) with acid in organic solvent or water or mixtures thereof;
b) neutralizing the obtained reaction mixture with base to precipitate out compound (IV);
c) condensing compound (IV) with molar excess of N-methyl piperazine carbonyl chloride (III) in presence of molar excess of base in a dipolar aprotic solvent to obtain racemic zopiclone (V);
d) reacting compound (V) with D(+)-O,O′-dibenzoyl tartaric acid monohydrate (VI) in acetonitrile and filtering the precipitated solid to isolate enantiomerically enriched D(+)-O,O′-dibenzoyl tartarate salt of zopiclone (VII);
e) purifying enantiomerically enriched D(+)-O,O′-dibenzoyl tartaric acid salt of zopiclone (VII) to get the pure tartarate salt of eszopiclone (VIII);
f) breaking the tartarate salt of eszopiclone (VIII) using base to get crude Eszopiclone (IX); and
g) purifying crude Eszopiclone (IX) to obtain pure Eszopiclone (I) substantially free of R-isomer (X).

15. The process as claimed in claim 13, wherein the acid is selected from organic or inorganic acid.

16. The process as claimed in claim 15, wherein the organic acid is selected from trifluoroacetic acid or trichloroacetic acid and inorganic acid is hydrochloric acid.

17. The process as claimed in claim 13, wherein the organic solvent is selected from dichloromethane, chloroform, ethylene dichloride, 1,1,1-trichloroethane, chlorobenzene, alcohol or mixture thereof.

18. The process as claimed in claim 17, wherein the alcohol is selected from methanol, ethanol or isopropanol.

19. The process as claimed in claim 13, wherein the base is selected from alkali metal carbonates or bicarbonates.

20. Eszopiclone obtained by the process as claimed in claim 1 having chemical purity of more than 99.5% and the unwanted (R)-isomer (X) and N-desmethylzopiclone (XI) being less than 0.1%.

21. The process as claimed in claim 14, wherein the acid is selected from organic or inorganic acid.

22. The process as claimed in claim 21, wherein the organic acid is selected from trifluoroacetic acid or trichloroacetic acid and inorganic acid is hydrochloric acid.

23. The process as claimed in claim 14, wherein the organic solvent is selected from dichloromethane, chloroform, ethylene dichloride, 1,1,1-trichloroethane, chlorobenzene, alcohol or mixture thereof.

24. The process as claimed in claim 23, wherein the alcohol is selected from methanol, ethanol or isopropanol.

25. The process as claimed in claim 14, wherein the base is selected from alkali metal carbonates or bicarbonates.
